(19) United States
(12) Plant Patent Application Publication
(10) Pub. No.: US 2002/0069438 P1
(43) Pub. Date: Jun. 6, 2002

(54) Sophora plant named 'Hilsop'

(76) Inventor: John Gifford Hillier, Romsey (GB)

Correspondence Address: MARK P. BOURGEOIS, P.O. BOX 95, OSCEOLA, IN, 46561, US

(21) Appl. No.: 09/728,687

(22) Filed: Dec. 2, 2000

(51) Int. Cl. A01H 5/00

(52) U.S. Cl. PLT/216

(57) ABSTRACT

A new cultivar of Sophora plant named ‘Hilsop’ that is characterized by increased hardiness, a vigorous habit, bushy form and long-lasting flowers that bloom four weeks earlier than all other existing varieties of Sophora.

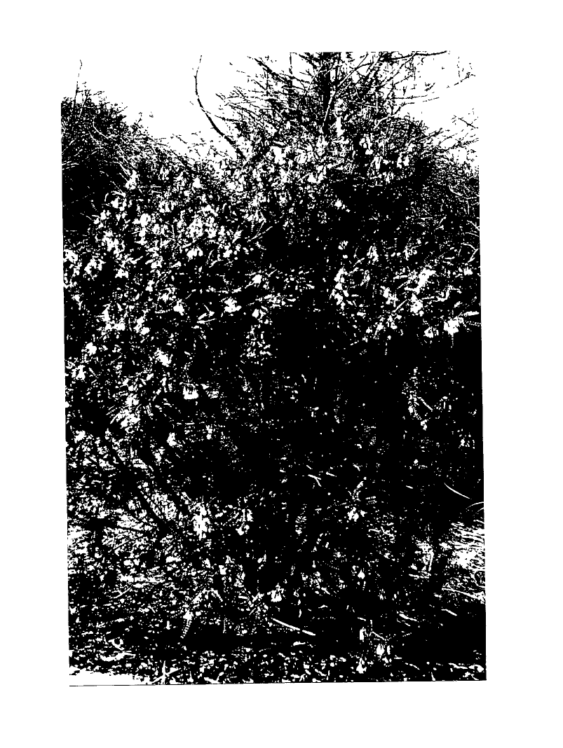

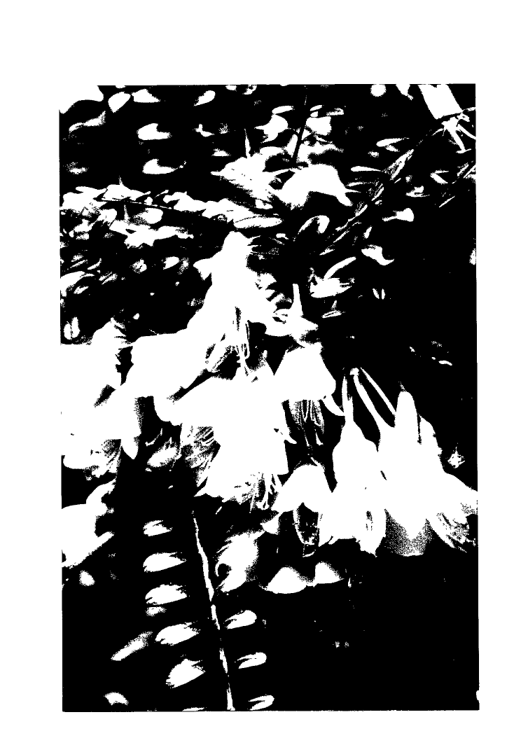

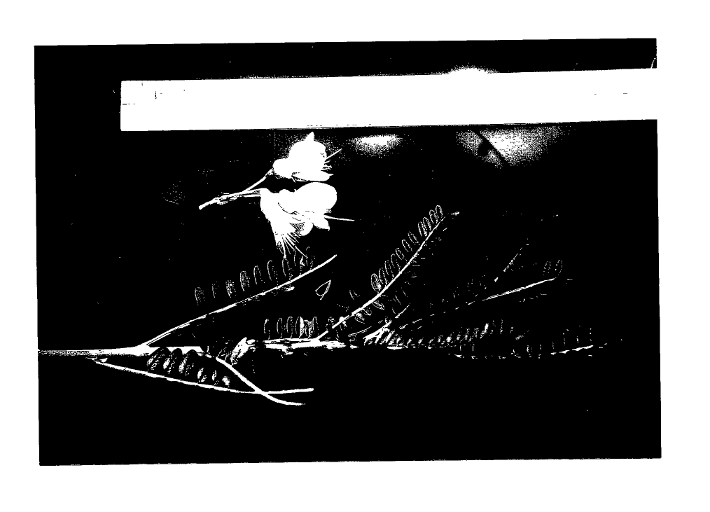

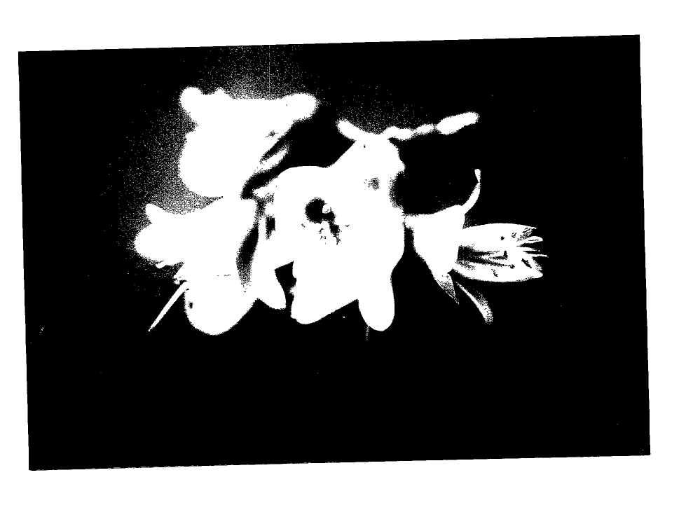

### BACKGROUND OF THE INVENTION

The present invention relates to a new and distinct cultivar known botanically as Sophora microphylla ‘Hilsop’ and referred to hereinafter by the cultivar name ‘Hilsop’. The new invention was discovered by the inventor in a cultivated area of Ampfield, Romsey, Hampshire United Kingdom. ‘Hilsop’ was a seedling derived from Sophora microphylla (unpatented) seed that was collected in Chile in 1982 and supplied by the Forestry Commission of England. The seed was sown by the inventor in 1982 at the Hillier Arboretum (now called the Sir Harold Hillier Gardens and Arboretum) and the resulting plants were observed for a period of time for their hardiness and general performance, before the inventor discovered the unique characteristics of ‘Hilsop’ in 1990.

The new invention, ‘Hilsop’, is distinguished from its parent by greater hardiness, earlier blooming, long lasting flowers, bushy form and vigorous growth. The closest comparison variety is Sophora microphylla (unpatented). In comparison ‘Hilsop’ has greater frost hardiness, having withstood approximately −18° Centigrade in the very hard winters of the late 1980's. In addition, ‘Hilsop’ is vigorous in growth, bushy in form and flowers four weeks earlier than its parent.

The new invention was first asexually propagated, by the inventor, in Romsey, Hampshire, United Kingdom in 1991. Since that time under careful observation, successive generations have been determined stable and uniform.

### SUMMARY OF THE INVENTION

The new Sophora named ‘Hilsop’ is distinguishable from all other known varieties of Sophora by the following characteristics:

1. Increased hardiness over the parent plant.

2. Vigorous habit.

3. Early blooming and long lasting yellow flowers.

4. Bushy form.

### BRIEF DESCRIPTION OF THE DRAWINGS

The accompanying color photographs illustrate the distinguishing traits of the new cultivar. Sheet 1 shows a side view of the entire plant, as it appears in The Sir Harold Hillier Gardens and Arboretum, and illustrates its foliage, flowers and habit. Sheet 2 is a close-up of the foliage and terminal racemes. Sheet 3 illustrates the size of leaflets and flower. Sheet 4 is a close-up of the inside of the flower, illustrating the pistil and stamens. All photographs are taken using conventional photographic techniques and although foliage colors may appear different from actual colors due to light reflectance, they are as accurate as possible by conventional photography.

### BOTANICAL DESCRIPTION OF THE PLANT

The following is a detailed description of the Sophora cultivar named ‘Hilsop’. Data was collected in Arroyo Grande, Calif. from plants grown in five-gallon containers and a twenty-four inch box specimen. Phenotypic differences may be observed with variations in environmental, climatic and cultural conditions, without however, any difference in genotype. Color determinations are in accordance with The Royal Horticultural Society Color Chart except where general color terms of ordinary dictionary significance are used. The growing requirements are similar to the species and no disease problems have been observed.

Botanical classification: Sophora microphylla ‘Hilsop’.

Parents: A seedling of Sophora microphylla (unpatented).

Common name: Kowhai.

Type: Large evergreen shrub or small evergreen tree.

Use: Border or lawn specimen.

Growth: Vigorous.

Habit: Upright and bushy.

Plant shape: Urn.

Height: 7.5 m. in height.

Width: 7.5 m. in width.

Branching habit: Deliquescent with older branches arching.

Flowering time: Diurnal.

Flowering season: March and April.

Hardiness: Resistant to frost and withstands −1820 Centigrade.

Seasonal interest: Profuse display of yellow flowers during spring.

Propagation method: Softwood semi-ripe cuttings and tissue culture.

Rooting period: Using rooting hormone, the initial cuttings require 4-6 weeks to begin showing roots and a further 2-3 weeks to be adequately established.

Optimum time for rooting: Late July and early August. Cuttings can be rooted as late as November, but with less success. When cuttings are taken after August, a bottom temperature of 20° Centigrade is maintained.

Soil: Grows well in any soil, irrespective of pH.

Light levels: Plant in full sunlight.

Cropping time: 1 year to 18 months.

Stem:

Main trunk diameter.—3.5 cm. in diameter.

Trunk color.—201A.

Surface of bark.—Mostly scabrous.

Secondary stem size.—0.75 cm. to 1.5 cm. in diameter and approximately 120 cm. in length.

Shape.—Cylindrical.

Internode length.—Ranges from 3 cm. to 7.5 cm. between nodes.

Branching arrangement.—Irregular, upright, interlocking branches.

Young stem color.—146C.

Young stem surface.—Sericeous.

Presence of markings.—Lenticels that are round or linear, and scarious stipules.

Size of stipules.—3-6 mm. in length and 3 mm. in width.

Stipule color.—201D.

Size of lenticels.—1-2 mm. in width and 1-2 mm. in length.

Lenticel color.—159A.

Foliage:

Leaf arrangement.—Alternate.

Leaflets.—Opposite bilaterally and distichous.

Division.—Pinnately compound.

Leaf appearance.—Fernlike.

Shape.—Elliptic to oblong.

Base.—Rounded.

Tip.—Rounded and occasionally retuse.

Venation.—Prominent mid-vein on upper and lower surfaces with dense fine hairs on undersurface.

Vein color (upper surface).—145A.

Vein color (lower surface).—145A.

Leaf type.—Evergreen.

Leaf surface (upper).—Mostly glabrous and slightly stippled. Hairs at base of mid-vein and sparsely on surface.

Leaf surface (lower).—Sericeous, appearing glabrous, but with presence of fine hairs.

Margins.—Entire.

Leaf length.—13 cm. in length.

Leaf width.—1 cm. to 1.5 cm. in width.

Leaflet length.—1.5 cm. in length.

Leaflet width.—0.50 cm. to 0.75 cm. in width.

Quantity.—Approximately 20 to 22 leaves per stem.

Young leaf color (upper).—189A.

Young leaf color (lower).—191A.

Mature leaf (upper).—Closest to 147A but darker.

Mature leaf (lower).—147B.

Opening leaf.—144A.

Petiole.—5 cm. in length and 3 mm. in width.

Petiolule.—1.5 cm. in length and 1 mm. in width.

Petiolule and petiole surface.—Hirsute.

Petiolule or petiole leaf mid-stem color.—Ranges from 197C on exposed surfaces and 199A,B,C on unexposed surface, to 146D particularly on young growth.

Foliar fragrance.—None observed.

Flower:

Shape.—Obliquely campanulate with tubular corolla tube.

Habit.—Pendulous.

Flower form.—Terminal racemes.

Flower arrangement.—Drooping cyme clusters.

Quantity of flowers.—3 to 7 per peduncle and approximately 50 racemes on a 24-inch box.

Fragrance.—None observed.

Flower longevity.—Approximately 3 weeks for a raceme.

Flower symmetry.—Assymetrical with a large petal and four smaller petals.

Sexuality.—Monecious.

Aspect.—Facing downward.

Flower diameter.—2 cm. in diameter.

Flower height.—3 cm. in height.

Self-cleaning or persistent.—Persistent.

Peduncle length.—2 cm. in length.

Peduncle diameter.—3 mm. in diameter.

Peduncle color.—197B to 199B.

Calyx form.—Tubular companulate.

Sepal color.—146D with veins 146B.

Sepal appearance.—Sericeous surface.

Sepal dimensions.—2 cm. in width and 1 cm in height.

Petals.—Five in number.

Petal length.—3 cm.

Petal width.—0.75 cm.

Fused or unfused.—Unfused.

Petal color (opening).—151A.

Petal color (mature).—Range of 153A,B, C with veins 153A.

Petal appearance.—Glabrous.

Corolla color.—14B with margins 12 A.

Petal color fully developed (outer surface).—12A.

Petal color fully developed (inner surface).—12A.

Bud color.—5A.

Unique features.—Veins running length of petal and one vexillum (large petal 3 cm. in length and 2 cm in width).

Reproductive organs:

Stamens.—Ten.

Stamen color.—Filament 153C, Anther 177A, B.

Pollen color.—17A.

Pistil length.—3.5 cm.

Style color.—145A.

Ovary.—non-visible.

Fruit:

None observed.

1. A new and distinct variety of sophora plant named ‘Hilsop’ as described and illustrated.
